(19) United States
(12) Patent Application Publication
(10) Pub. No.: US 2025/0057559 A1
(43) Pub. Date: Feb. 20, 2025

(54) ENDOSCOPIC SURGICAL DEVICES AND METHODS OF USING THE SAME

(71) Applicant: University of Washington, Seattle, WA (US)

(72) Inventors: Ché ROSS, Seattle, WA (US); Joseph BIGHAM, Seattle, WA (US)

(21) Appl. No.: 18/803,366

(22) Filed: Aug. 13, 2024

Related U.S. Application Data
(60) Provisional application No. 63/532,739, filed on Aug. 15, 2023.

(51) Int. Cl. A61B 17/29; A61B 17/00

(52) U.S. Cl. CPC A61B 17/2909; A61B 2017/00367; A61B 2017/00477; A61B 2017/00561; A61B 2017/2939; A61B 2217/005

(57) ABSTRACT

Apparatuses, systems, and methods are disclosed for a surgical device with suction capabilities to effectively remove fluid, tissue, and other debris from a surgical site and with a gripper for manipulating tissues and other anatomical structures during surgery. The surgical device also includes a handle having an actuator operable for controlling a position of the gripper and a suction control for adjusting a suctioning level of the device as desired, where the actuator and suction control are arranged on the handle to facilitate individual or simultaneous operation of both mechanisms.

### CROSS REFERENCE TO RELATED APPLICATION

This application claims priority benefit under 35 U.S.C. § 119 (c) of U.S. Provisional Patent Application No. 63/532,739, filed Aug. 15, 2023, the disclosure of which is hereby incorporated by reference in its entirety.

### TECHNICAL FIELD

The present disclosure relates generally to the field of medical devices. In particular, the disclosure relates to endoscopic surgical instruments with suctioning and grasping functionalities to facilitate surgery.

### BACKGROUND

Tissues in the body may be treated in surgical or medical procedures using various techniques and surgical devices, such as for tissue resection and removal. During surgical procedures, manipulation and removal of debris, blood and other bodily fluids, and/or tissues is typically required to maintain appropriate visibility of the surgical site and to help ensure a successful surgery. During endoscopic surgeries in general, obtaining hemostasis (the prevention and control of bleeding) is critical for providing adequate anatomical visualization at the surgical site and improving surgical outcomes. Moreover, the reduced scope of the surgical field-of-view in such surgeries as compared to open surgeries drastically increases the importance of reducing intraoperative bleeding. While there are preoperative/intraoperative methods that may help reduce bleeding, novel technologies and techniques to improve visualization of the surgical field are paramount from a surgical perspective.

Despite recent advances in the design of endoscopic surgical instruments and improvements in surgical techniques overall, conventional surgical devices are ineffective at accomplishing these tasks. For example, current surgical devices lack a multi-functional design that offers the capability of providing suctioning, ligating, and grasping functions in a single device. Accordingly, surgeons must be adept at maneuvering and juggling between various surgical instruments at once, which not only increases surgical timelines (and the associated risk of infection) but also crowds the surgical site with multiple tools. While many surgeons may be capable of managing multiple instruments at a time, the concurrent use of various surgical instruments at a surgical site makes it challenging for a surgical assistant to participate in some microscopic/endoscopic surgeries due to the limited scope of the surgical field.

In the field of endoscopic endonasal surgery, a minimally invasive method that allows a surgeon to go through the nasal cavity to operate on areas of the brain and the spine, cerebrospinal fluid (CSF) leakage and bleeding are major risk factors that impact the overall rates of successful treatment and patient outcomes. To avoid CSF leakage and obtain hemostasis, surgeons must use appropriate techniques for proper closure/ligation. In the case of repairing CSF leaks, a fascial inlay patch suture may be used, but that requires suturing dural incision while holding the fascia flap in the intracranial epi-arachnoid space-thereby requiring a constant interchange between needle holders and scissors/suction. Moreover, visualization of the surgical site can be made difficult with the presence of a persistent CSF leak or hemorrhage.

### SUMMARY

Various examples of medical devices for endoscopic surgical treatments are described herein. An example device may include a handle having a first end and an opposite second end and an elongated body extending outwardly from the second end of the handle. The device includes a gripper having at least a portion thereof extending outwardly from a distal end face of the elongated body and an actuator operable to control a position of the gripper. A suction conduit is operably coupled along the first end of the handle and is configured to deliver a vacuum air flow through the handle and the elongated body to draw fluid, tissue, or other debris toward the elongated body. The handle includes a suction control operable to adjust a flow rate of the vacuum air flow delivered through the handle and the elongated body.

In some examples, the vacuum air flow may be directed through a first pathway of the elongated body when the gripper is in an open position, and through a second pathway of the elongated body when the gripper is in a closed position. In some examples, the gripper may include a first gripper segment and a second gripper segment, and the vacuum air flow through the elongated body flows between the first gripper segment and the second gripper segment when the gripper is in the open position.

It should be understood that the foregoing summary provides certain examples further described herein and is not intended to identify any key or critical aspects of the disclosed or claimed subject matter. Further, aspects of the example systems summarized above may be combined in any suitable manner without departing from the principles of the disclosed or claimed subject matter.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several examples in accordance with the disclosure and are therefore not to be considered limiting in scope, the disclosure will be described with additional specificity and detail through use of the accompanying drawings, in which:

FIG. 1 is a schematic illustration of a surgical device in accordance with examples described herein.

FIG. 2 is an enlarged view of a distal portion of the surgical device of FIG. 1 illustrating a gripper in a closed position in accordance with examples described herein.

FIG. 3 is a cross-section view of a handle of the surgical device of FIG. 1 illustrating an actuator for controlling a position of the gripper in accordance with examples described herein.

FIG. 4 is a schematic illustration of another surgical device in accordance with examples described herein.

FIG. 5 is an enlarged view of a distal portion of the surgical device of FIG. 4 illustrating a gripper in a closed position in accordance with examples described herein.

### DETAILED DESCRIPTION

Certain details are set forth below to provide a sufficient and clear understanding of example embodiments of the disclosure. However, it will be clear to one skilled in the art that embodiments of the disclosure may be practiced without some particular details. Moreover, the particular embodiments of the present disclosure described herein are provided by way of example and should not be used to limit the scope of the claimed subject matter to these particular embodiments. In other instances, well-known materials, components, processes, and/or anatomy have not been described or shown in detail in order to avoid unnecessarily obscuring the pertinent details of the embodiments.

This disclosure is directed to a multi-functional, endoscopic or microscopic device designed for performing surgery at a targeted surgical site. In one example use, the disclosed device may be used for endoscopic/microscopic surgeries where a suction device and gripper device for tissue are needed, especially when working with a narrow field-of-view. For example, in some embodiments, the device disclosed herein has a preferred use for endoscopic endonasal surgeries to access various areas of the brain and spine, such as for transsphenoidal pituitary resection to remove pituitary and other intrasellar tumors. In such examples, the device may be introduced via an endoscope through a patient's nasal cavity and advanced to reach the surgical site. As further described in detail below, the device generally functions as a suction instrument having a vacuum air flow operable to draw debris and fluid away from the surgical site to improve visualization. The device also includes a gripper, which may include a plurality of teeth or other engagement features thereon, wherein the gripper extends outwardly along a distal end of the device to aid in grasping, grappling, gripping, and/or manipulating tissue or other anatomical structures at the surgical site. The dual suctioning and grasping functionalities of the device minimizes the need for surgeons and other medical personnel to switch between various instruments during surgery, thereby expediting surgical procedures and improving patient outcomes.

In some embodiments, the device further includes a suction control on a handle portion thereof to allow for customization and control of suction strength via the device. In some embodiments, the device also includes an actuator, such as a lever, switch, or other suitable control mechanism, that allows a surgeon or other medical personnel to easily and conveniently operate the gripper during surgery. In one or more embodiments, the device or components thereof may comprise surgical steel, nitinol, and/or any other suitable and durable biocompatible material.

As described, the device disclosed herein aims to increase dexterity and allow for micro-movements of the device at the surgical site to improve surgical techniques, both of which are beneficial in endoscopic/microscopic surgeries that take place in areas of the body with narrow fields-of-view and involving delicate anatomy. In some embodiments of the present disclosure, the device may help reduce or minimize bleeding time until homeostasis is obtained and help avoid complications caused by cerebrospinal fluid (CSF) leaks while improving the overall field-of-view at the surgical site for surgeons. In other embodiments, the device may be used for surgeries and treatment with any of a variety of tissue sites and anatomical structures other than those described herein. Additional aspects of these and other example systems will be apparent from the following detailed description of example embodiments, which proceed with reference to the accompanying drawings.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

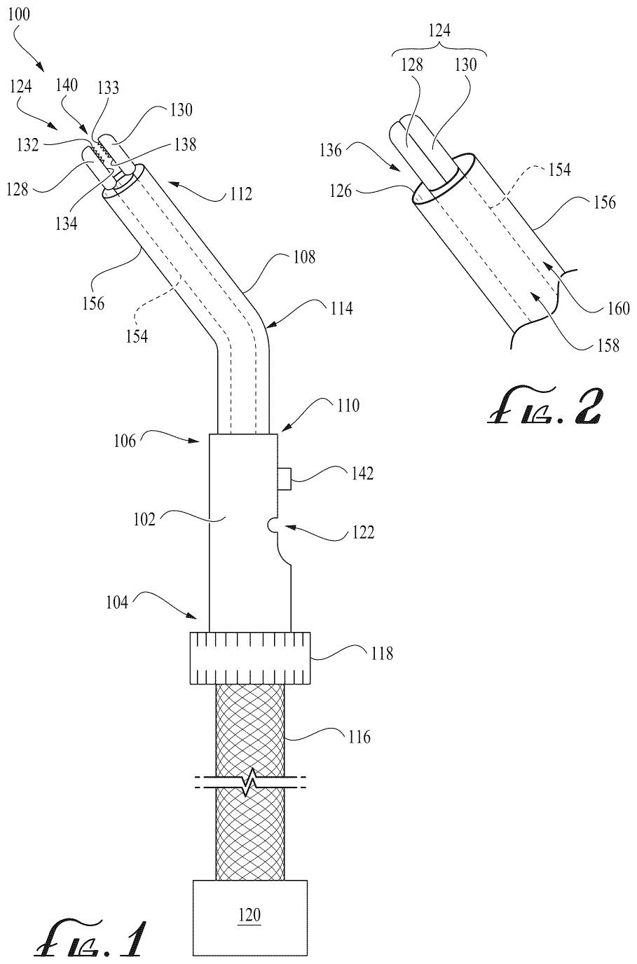

With collective reference to FIGS. 1-5, the following describes a surgical device 100, 200 designed for endoscopic delivery to a targeted surgical site for treatment, where the device 100, 200 is configured to provide controlled suctioning functionality at the surgical site and facilitate grasping and manipulating of tissues and/or other anatomical structures at the surgical site. As noted previously, this dual-functionality of the surgical device 100, 200 helps optimize the surgical field-of-view by reducing the number of surgical tools required to complete treatment at the surgical site, which also helps expedite surgeries and improve overall patient outcomes. The device 100, 200 also allows for customized control of suctioning capabilities to help remove bodily fluids and debris as needed from the surgical site to facilitate treatment. The following provides additional details of the device 100, 200 in accordance with example embodiments.

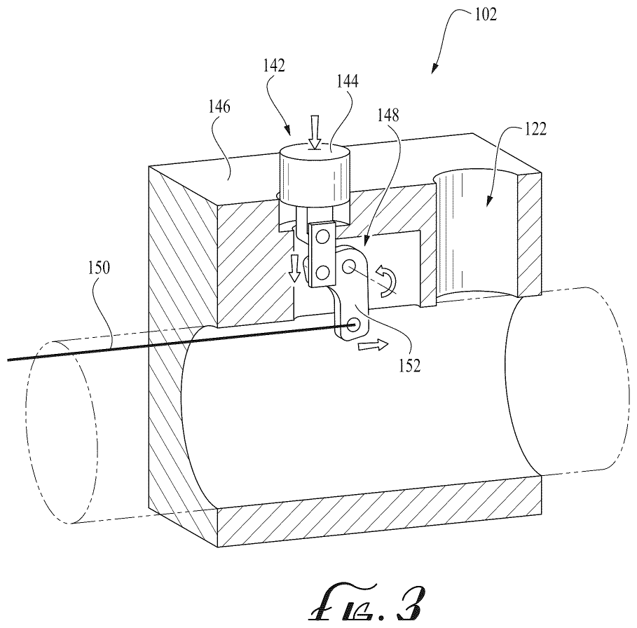

FIG. 1 is a schematic illustration of a surgical device 100 arranged in accordance with example embodiments described herein. With reference to FIG. 1, the device 100 includes a handle 102 with a first (proximal) end 104 and an opposite second (distal) end 106. The device 100 further includes an elongated body 108 extending outwardly from the distal end 106 of the handle 102. The elongated body 108 includes a first (proximal) portion 110 and an opposite second (distal) portion 112, where the proximal portion 110 is adjacent and extends outwardly from the distal end 106 of the handle 102. In some embodiments, the elongated body 108 is removably coupled to the distal end 106 of the handle 102, while in other embodiments, the elongated body 108 may be formed as an integral portion of the handle 102 and designed not to be removable therefrom. In some embodiments, the elongated body 108 may include a bent or curved region 114 between the proximal portion 110 and the distal portion 112 of the elongated body 108. The bent or curved region 114 of the device 100 may help aid navigation and delivery of the device 100 through nasal or other cavities of the patient to reach the surgical site. The elongated body 108 includes a distal opening 136 (see FIG. 2) having a diameter ranging from approximately 1.5 mm to 4.0 mm. In other embodiments, the diameter of the distal opening 136 may be different than the aforementioned range.

As illustrated in FIG. 1, the surgical device 100 further includes a suction conduit 116, such as a tube, pipe, or other suitable element having an interior cavity (not shown) designed for conveying fluids therethrough. The suction conduit 116 is operably coupled to the handle 102 along its proximal end 104. In some embodiments, the surgical device 100 may further include a connector 118 disposed along the proximal end 104, and the suction conduit 116 may be coupled to the connector 118. The connector 118 may be integrally formed as a portion of the handle 102 or it may otherwise be coupled to the handle 102 via any suitable method (e.g., threaded, press fit, etc.). In these configurations, an opposite end (not shown) of the suction conduit 116 is coupled to a vacuum pump or other suitable suction source 120 capable of providing a vacuum air flow to create the suction functionality of the surgical device 100. For example, the suction conduit 116 delivers a vacuum air flow through the handle 102 and the elongated body 108 to draw fluid, tissue, or other debris from the surgical treatment site toward the elongated body 108 during surgery.

In some embodiments, the handle 102 may further include a suction control 122 disposed between its proximal end 104 and distal end 106, where the suction control 122 is operable to adjust a flow rate of the vacuum air flow delivered through the handle 102 and the elongated body 108. In one embodiment, the suction control 122 may include an aperture (see FIG. 3) formed along the handle 102, where obstruction of the aperture adjusts the flow rate of the vacuum air flow. For example, a surgeon holding the handle 102 may use a thumb or finger to increase or decrease the flow rate by varying the degree to which the aperture is covered or uncovered. In some embodiments, the surgeon may reduce the flow rate by uncovering more of the aperture and may increase the flow rate by covering more of the aperture. In other embodiments, an external plug or resilient seal (not shown) may instead be used to operate the suction control 122. It should be understood that the foregoing is simply an example embodiment of a suction control 122. In other embodiments, other designs may be used to adjust the suction flow rate of the surgical device 100.

As illustrated in FIG. 1, the surgical device 100 further includes a gripper 124 operable to grasp and/or manipulate tissues and other anatomical structures at the surgical site. In one embodiment, the gripper 124 may be flush relative to and extend outwardly from a distal end face 126 (see FIG. 2) of the second portion 112 of the elongated body 108 through the distal opening 136. In one example embodiment, the gripper 124 may extend outwardly from the distal end face 126 by approximately 1 mm to 5 mm. In other example embodiments, the gripper 124 may extend further than 5 mm from the distal end face 126 depending on a desired depth for a particular surgical site. The gripper 124 may include a first gripper segment 128 and a second gripper segment 130, where each of the gripper segments 128, 130 includes one or more grip elements 132, 133 disposed on a surface thereof to aid with gripping tissues and anatomical structures during surgery. In some embodiments, the gripper 124 may have a width of between 1.5 mm to 4.0 mm spanning from an outer peripheral edge of the first gripper segment 128 to an outer peripheral edge of the second gripper segment 130. In other embodiments, the width of the gripper 124 may be larger than 4.0 mm.

The one or more grip elements 132, 133 may include any suitable feature for improving grip of the gripper 124, such as teeth or grooves having any one of various suitable geometries, including sawtooth, incisor, molar, serrated, ribbed, curved. For example, in one embodiment, the first gripper segment 128 may include one or more grip elements 132 comprising a plurality of teeth formed on an interior surface 134 thereof. Similarly, the second gripper segment 130 may include one or more grip elements 133 comprising a plurality of teeth formed on an interior surface 138 thereof. As illustrated in FIG. 1, the respective interior surfaces 134, 138 are arranged such that they face one another. The one or more grip elements 132, 133 may be designed with various features as desired to accomplish various goals as noted previously. For example, in some embodiments, the one or more grip elements 132, 133 may be serrated or beveled teeth to help improve grasping of tissues during surgery, while in other embodiments, the one or more grip elements 132, 133 may be dull or flattened ribs or other features to minimize trauma or damage caused by the gripper 124 during surgery. In still other embodiments, the one or more grip elements 132, 133 may be curved or recessed to help grasp and remove resected tissue or other anatomical structures from the surgical site. In still other embodiments, the gripper 124 may include other suitable configurations.

The gripper 124 is adjustable to an open position (see FIG. 1), whereat the first gripper segment 128 is spaced apart from the second gripper segment 130, with a gap 140 defined therebetween. When the gripper 124 is moved to the closed position (see FIG. 2), the first gripper segment 128 contacts the second gripper segment 130. In an embodiment where the gripper segments 128, 130 each include one or more grip elements 132, 133 (e.g., a plurality of teeth) formed thereon, the one or more grip elements 132, 133 may align relative to one another to allow the gripper 124 to be tightly closed (i.e., the gap 140 is substantially or entirely eliminated as shown in FIG. 2).

In one embodiment, the position of the gripper 124 may be controlled via an actuator 142 that is operably coupled to the gripper 124. The actuator 142 is preferably disposed on the handle 102 between its proximal end 104 and distal end 106 in a suitable location to allow the operator of the device 100 to comfortably access one or both of the actuator 142 and the suction control 122 with one or more digits while holding the device 100 in one hand. For example, in one embodiment, the suction control 122 is arranged at an approximate midpoint of the handle 102, and the actuator 142 is disposed between the suction control 122 and the distal end 106 of the handle 102 to allow an operator to use a thumb (or combination of other digits) to both adjust flow rate via the suction control 122 and to manipulate the actuator 142 to control a position of the gripper 124. The actuator 142 may take any of several suitable forms and mechanisms operable for driving a position of the gripper 124. For example, the actuator 142 may be a sliding lever or button (as shown in the example of FIG. 3), a rotating wheel, a pneumatic push button, a toggle button, or any other suitable component that would enable a means of opening and closing the gripper 124.

With reference to FIG. 3, the following provides an example embodiment of an actuator 142 in accordance with one embodiment. FIG. 3 is an exploded, cross-section view of the handle 102 of the surgical device 100 in accordance with an embodiment. With reference to FIG. 3, the actuator 142 includes a depressible element 144 (such as a button or a tab) extending outwardly from an exterior surface 146 of the handle 102. In one embodiment, the element 144 is manually actuatable to drive a linkage system 148 for operating the gripper 124. The linkage system 148 may include a line or cable 150 coupled to a link 152 on one end of the line 150 and the gripper 124 on another end (not shown). In one embodiment, the line or cable 150 extends from the link 152 through an interior of the elongated body 108 to couple with the gripper 124.

When the element 144 is manually depressed downwardly, the portion of the link 152 of the linkage system 148 to which the line 150 is coupled is driven generally rearwardly to pull the line 150 rearwardly and adjust the gripper 124 to an open position (see FIG. 1) by moving one or both gripper segments 128, 130. The gripper 124 may be moved from the open position to the closed position (see FIG. 2) by releasing the element 144, which in turn allows the link 152 to return to its initial position and release tension on the line 150. The release of tension on the line 150 causes the gripper 124 to return to its closed position. In one embodiment, the line 150 may be coupled to only one of the first gripper segment 128 or the second gripper segment 130 while the other of the first gripper segment 128 or the second gripper segment 130 remains stationary. In such embodiments, the actuator 142 adjusts a position of only one of the gripper segments 128, 130 (i.e., the gripper segment 128, 130 coupled to the line 150) relative to the other (i.e., the gripper segment 128, 130 not coupled to the line 150) to control operation of the gripper 124. It should be understood that in other embodiments, both gripper segments 128, 130 may be independently operable via the actuator 142 (or via multiple actuators).

In some embodiments, a flow pathway for the vacuum air flow from the suction source 120 through the elongated body 108 and outward relative to the distal end face 126 may be determined based on a position of the gripper 124. For example, with reference to FIGS. 1-2, in one embodiment, the elongated body 108 may include an interior conduit 154 and an exterior conduit 156 surrounding the interior conduit 154, where the conduits 154, 156 may include a tube, pipe, or other suitable hollow body through which vacuum air flow may be directed. The interior conduit 154 is substantially hollow and defines an interior suction passage 158 in operable communication with the suction conduit 116. Similarly, the exterior conduit 156 is substantially hollow and defines an exterior suction passage 160 in operable communication with the suction conduit 116.

In these embodiments, when the gripper 124 is in the open position (see FIG. 1), the vacuum air flow through the elongated body 108 flows through the interior conduit 154 along the interior suction passage 158 and out through distal end face 126 of the elongated body 108. As illustrated in FIG. 1, the gripper segments 128, 130 in the open position substantially obstruct a portion of the distal end face 128 and restrict flow of the vacuum air flow outwardly of the elongated body 108 through the exterior conduit 156 along the exterior suction passage 160. When the gripper 124 is in the open position, the vacuum air flow draws tissues and/or other anatomical structures toward the elongated body 108 and between the gripper segments 128, 130 (i.e., along the gap 140), which may aid with grasping and/or manipulating the drawn tissues and/or anatomical structures.

Similarly, when the gripper 124 is in the closed position (see FIG. 2), the vacuum air flow through the elongated body 108 flows through the exterior conduit 156 along the exterior suction passage 160 and out through distal end face 126 of the elongated body 108 along the distal opening 136. As illustrated in FIG. 2, the gripper segments 128, 130 in the closed position substantially obstruct a portion of the distal end face 126 and restrict flow of the vacuum air flow outwardly of the elongated body 108 through the interior conduit 156 along the interior suction passage 158. In some embodiments, when the gripper 124 is in the closed position, the vacuum air flow may be used to draw bodily fluids (such as blood, CSF, etc.) into the elongated body 108 and away from the surgical site.

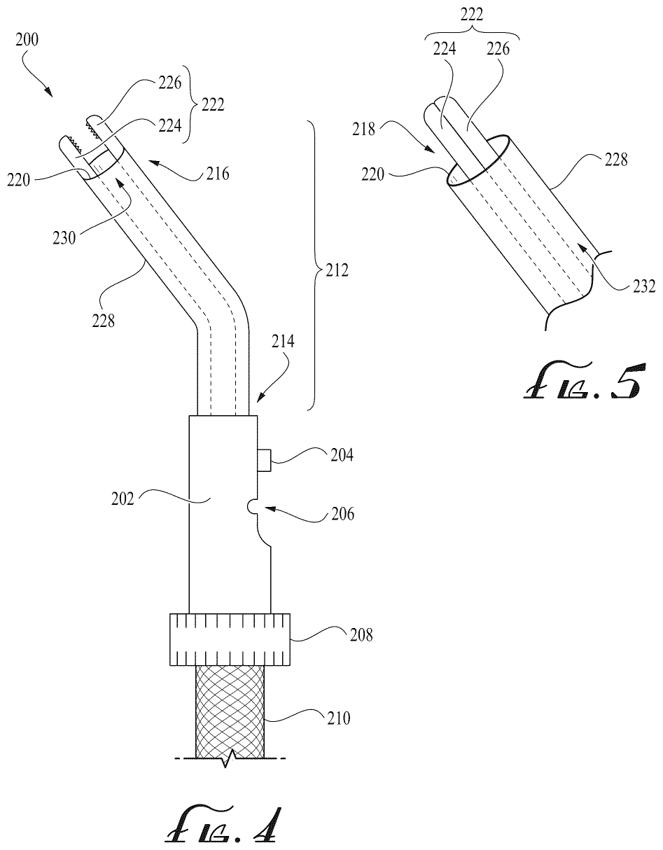

FIG. 4 illustrates a surgical device 200 in accordance with another example embodiment. The surgical device 200 includes many of the same components as the device 100 arranged in a similar manner and with similar functionality as described previously. For example, the device 200 includes a handle 202 with an actuator 204 and a suction control 206 each of which operates in a similar manner as previously described with reference to actuator 142 and suction control 122 of the device 100 of FIG. 1. The handle 202 further includes a connector 208 coupled thereto, the connector 208 receiving a suction conduit 210 operable to deliver a vacuum air flow in a similar fashion as described previously. Additional details of these and other like components of the device 200 are not further described in detail herein to avoid obscuring more pertinent features of the embodiment with the understanding that like features and components illustrated in FIG. 4 for the device 200 operate in a similar fashion as the respective features and components described with reference to device 100.

With reference to FIG. 4, the device 200 includes an elongated body 212 having a first (proximal) end 214 coupled to the handle 202, and a second (distal) end 216 with a suction opening 218 on a distal end face 220. The elongated body 212 houses a gripper 222 having a first gripper element 224 and a second gripper element 226, wherein the gripper 222 is controllable via the actuator 204 to move the gripper 222 between an open position and a closed position in a similar fashion as previously described with reference to device 100. As illustrated in FIG. 4, the elongated body 212 includes a sheath or covering 228 with the gripper 222 disposed at least partially therein, the gripper elements 224, 226 extending outwardly from the distal end face 220. In some embodiments, the gripper 222 may extend substantially through the entirety of the covering 228, while in other embodiments, the gripper 222 may only extend partially through the covering 228. When the gripper 222 is in the open position, the vacuum air flow through the elongated body 212 flows through a first pathway 230 and outwardly through the suction opening 218 on the distal end face 220 between the first gripper segment 224 and the second gripper element 226 (see FIG. 4). When the gripper 222 is in the closed position, the vacuum air flow through the elongated body 212 flows through a second pathway 232 and outwardly through the suction opening 218 on the distal end face 220 (see FIG. 5). In a similar fashion as described previously, the suction control 206 allows a surgeon or other medical personnel to adjust a flow rate of the vacuum air flow through the pathways 230, 232, and the open or closed position of the gripper 222 determines which of the pathways 230, 232 the vacuum air flow passes. In some embodiments, the vacuum air flow may flow continuously through the elongated body 212 via the pathways 230, 232.

In other embodiments, the devices 100, 200 may include other features or systems for controlling the air flow pathways of the vacuum air flow. For example, in other embodiments, the devices 100, 200 may include one or more valves (not shown) that may be opened and closed to direct vacuum air flow through the elongated body between various pathways. In some embodiments, the one or more valves may be operated via the actuator (e.g., actuator 142, 204) such that when the actuator is used to open the gripper (e.g., gripper 124, 222), a valve opens to direct vacuum air flow through a first pathway that directs the flow outwardly of the elongated body between the gripper elements. Similarly, when the actuator is released, the valve closes to restrict air flow through the first pathway and instead direct air flow through a second pathway such that it flows outwardly of the elongated body and around an exterior surface of the gripper. In other embodiments, other configurations are possible without departing from the principles of the disclosed subject matter.

In some embodiments, the surgical devices 100, 200 described herein may be coupled and advanced to the surgical site via another flexible device (not shown) such as a catheter, an endoscope, or other suitable medical equipment operable to navigate the nasal cavity or other suitable cavity. In some embodiments, the surgical devices 100, 200 may be guided manually or via a robotically-assisted guidance systems, or other suitable systems.

It should be understood that example embodiments provided herein of both the design of the surgical device and any potential clinical applications associated therewith are not intended to be limiting. Other configurations of the surgical device, as well as different surgical applications that would benefit from the use of the disclosed subject matter, are possible and encompassed within the scope of the disclosure. In addition, it is to be appreciated that any one of the above embodiments or processes, or specific features associated therewith, may be combined with one or more other embodiments and/or processes or be separated and/or performed amongst separate devices or device portions in accordance with the present systems, devices, and methods. Further, while some advantages associated with certain embodiments of the disclosure may have been described in the context of these embodiments, other embodiments may also exhibit such advantages or may exhibit different advantages. It should be understood that not all embodiments need necessarily exhibit such advantages to fall within the scope of the claimed subject matter.

Finally, the disclosure is intended to be merely illustrative of the present devices, apparatuses, systems, and methods and should not be construed as limiting the appended claims to any particular embodiment or group of embodiments. Thus, while the present disclosure has been described in particular detail with reference to exemplary embodiments, it should also be appreciated that numerous modifications and alternative embodiments may be practiced without departing from the broader and intended spirit and scope of the present disclosure as set forth in the claims that follow. Accordingly, the specification and drawings are to be regarded in an illustrative manner and are not intended to limit the scope of the appended claims.

1. A surgical device comprising:
a handle having a first end and an opposite second end;
an elongated body having a first portion and an opposite second portion, the first portion of the elongated body extending outwardly from the second end of the handle;
a gripper having at least a portion thereof extending outwardly from a distal end face of the second portion of the elongated body;
an actuator disposed between the first end and the second end of the handle, the actuator operably coupled to the gripper, wherein the actuator is operable to control a position of the gripper;
a suction conduit operably coupled along the first end of the handle, the suction conduit configured to deliver a vacuum air flow through the handle and the elongated body to draw fluid, tissue, or other debris toward the elongated body; and
a suction control disposed between the first end and the second end of the handle, the suction control operable to adjust a flow rate of the vacuum air flow delivered through the handle and the elongated body.

2. The surgical device of claim 1, further comprising a connector disposed on the first end of the handle, wherein the suction conduit is removably coupled to the connector.

3. The surgical device of claim 1, wherein the gripper includes a first gripper segment and a second gripper segment, and wherein the actuator is operable to move the gripper into an open position, whereat the first gripper segment is spaced apart from the second gripper segment, and wherein the actuator is operable to move the gripper into a closed position, whereat the first gripper segment contacts the second gripper segment.

4. The surgical device of claim 3, wherein the vacuum air flow through the elongated body flows through a first pathway when the gripper is in the open position, and wherein the vacuum air flow through the elongated body flows through a second pathway when the gripper is in the closed position.

5. The surgical device of claim 3, wherein the vacuum air flow through the elongated body flows between the first gripper segment and the second gripper segment when the gripper is in the open position.

6. The surgical device of claim 1, the gripper including a first gripper segment and a second gripper segment movable relative to another, wherein the first gripper segment includes a first plurality of grip elements formed on a first surface thereof, and the second gripper segment includes a second plurality of grip elements formed on a second surface thereof, wherein the first and second surfaces face one another.

7. The surgical device of claim 1, wherein the elongated body includes a first suction passage and a second suction passage for the vacuum air flow, and wherein when the gripper is in a first position, the first suction passage is open and the second suction passage is closed, and wherein when the gripper is in a second position, the first suction passage is closed and the second suction passage is open.

8. The surgical device of claim 7, the gripper including a first gripper segment and a second gripper segment, wherein the first position of the gripper is an open position whereat the first gripper segment and the second gripper segment are offset from one another, and wherein the second position of the gripper is a closed position whereat the first gripper segment and the second gripper segment contact one another.

9. The surgical device of claim 1, wherein the suction control further includes a suction aperture formed on the handle, and wherein the flow rate of the vacuum air flow through the elongated body is adjustable based on an obstruction of the suction aperture.

10. The surgical device of claim 1, wherein the actuator is disposed between the suction control and the second end of the handle.

11. The surgical device of claim 1, wherein the actuator extends outwardly from an exterior surface of the handle and includes a line coupled to the gripper, and wherein the actuator is manually actuatable to adjust the line to control the position of the gripper.

12. The surgical device of claim 11, wherein the gripper automatically returns to an initial position in response to a release of the actuator.

13. The surgical device of claim 11, wherein the actuator includes an element extending outwardly from an exterior surface of the handle, the element movable between a first position and a second position, and wherein the gripper is in an open position when the element is in the first position and a closed position when the element is in the second position.

14. The surgical device of claim 1, wherein the gripper extends into at least a portion of the elongated body.

15. The surgical device of claim 1, wherein the elongated body further includes an interior conduit and an exterior conduit surrounding the interior conduit, wherein the interior conduit includes an interior suction passage and the exterior conduit includes an exterior suction passage for the vacuum air flow through the elongated body.

16. The surgical device of claim 15, wherein when the gripper is in a closed position, the vacuum air flow through the elongated body flows through the exterior suction passage, and wherein when the gripper is in an open position, the vacuum air flow through the elongated body flows through the interior suction passage.

17. The surgical device of claim 16, wherein when the gripper is in the closed position, the vacuum air flow is restricted from flowing outwardly of the elongated body through the interior suction passage.

18. The surgical device of claim 16, wherein when the gripper is in the open position, the vacuum air flow is restricted from flowing outwardly of the elongated body through the exterior suction passage.

19. The surgical device of claim 1, wherein the elongated body is formed as an integral portion of the handle.

20. The surgical device of claim 1, wherein the elongated body is removably coupled to the second end of the handle.
